(19) United States
(12) Patent Application Publication
(10) Pub. No.: US 2025/0044264 A1
(43) Pub. Date: Feb. 6, 2025

(54) METHOD FOR DETERMINING THE STANDARDIZED SOOTING TENDENCY OF A SUBSTANCE MIXTURE AND REPRESENTATIVE INDICES FOR CALCULATING AN OVERALL SOOTING TENENCY

(71) Applicant: Volkswagen Aktiengesellschaft, Wolfsburg (DE)

(72) Inventors: Yvonne BITTNER, Braunschweig (DE); Kerstin KOERTKE, Hannover (DE)

(73) Assignee: Volkswagen Aktiengesellschaft, Wolfsburg (DE)

(21) Appl. No.: 18/920,854

(22) Filed: Oct. 19, 2024

Related U.S. Application Data
(63) Continuation of application No. PCT/EP2023/061117, filed on Apr. 27, 2023.

(30) Foreign Application Priority Data
Apr. 29, 2022 (DE) 10 2022 110 522.9

(51) Int. Cl. G01N 30/86; G01N 30/02

(52) U.S. Cl. CPC G01N 30/8675; G01N 2030/025

(57) ABSTRACT

A method for determining the standardized sooting tendency of a substance mixture and representative indices for calculating an overall sooting tendency. The sooting tendency is determined using gas-chromatographic component analysis of hydrocarbon types and oxygenates of a substance mixture, in particular of a fuel or of an additive via a reformulyzer analysis. A standardized total yield sooting index) of substance mixtures according to YSI_{Total}=ΣYSIᵢ·Vᵢ/100 is determined, wherein the volumetric proportion (Vᵢ) of each reformulyzer subgroup (i) is multiplied by that of the yield sooting index (YSIᵢ) associated with the reformulyzer subgroup (i), according to which the determined values are summated to the total yield sooting index (YSI_{Total}). Furthermore, method variants are described for determining yield sooting indices (YSIᵢ-M, YSIᵢ-Mid, YSIᵢ-GewM) representing the yield sooting index (YSIᵢ) associated with the subgroups (i).

This nonprovisional application is a continuation of International Application PCT/EP2023/061117, which claims priority to German Patent Application No. 10 2022 110 522.9, which was filed in Germany on Apr. 29, 2022, and which are both herein incorporated by reference.

### BACKGROUND OF THE INVENTION

### Field of the Invention

The invention relates to a method for determining a sooting tendency using gas-chromatographic component analysis of hydrocarbon types and oxygenates of a substance mixture.

### Description of the Background Art

Today, the topic of soot particle formation of fuels is very critical since the emissions or the soot particle formation of motor vehicles is to be reduced further. This is associated in particular with the tightening of directives in connection with the EU 7 reform. The new directives must be met for the motor vehicles or for the fuels, and a range of different fuels must be tested. Furthermore, the supply efficacy must be checked, for example, for city fuels. The YSI (Yield Sooting index) is a good basis for describing the sooting tendency. In German, the YSI is referred as the ‘Ruβbildungsneigungs-Index’. This index is currently complicated to determine. According thereto, it is possible to determine the yield sooting index by means of an individual component analysis of a fuel. Disadvantageously, an individual component analysis must thus be carried out when determining the sooting index. This analysis involves a lot of data and is very time consuming and cost intensive. Furthermore, there is the problem that in this method not all components can be assigned and are listed as unknown. Disadvantageously, this ultimately results in deviations in the calculation.

DE 689 27 133 T2 is known from the prior art. It proposes a method for determining the aromatic carbon content of a hydrocarbon-containing solution which contains more than one aromatic compound, in which method the procedure is as follows. (a) The solution is separated into fractions by means of liquid chromatography. (b) Each fraction is irradiated separately with UV light in a wavelength range of which at least a portion is in the range from 200 nm to 400 nm. (c) The absorption of the UV light is measured by the aromatic hydrocarbons in each fraction. (d) The integral of the absorption is then determined as a function of the photon energy over the energy corresponding to the wavelength range. (e) Finally, the aromatic carbon content is determined from the absorption integral.

EP 1 953 545 A1, which corresponds to US 2008/0180447, and which discloses a method for the quantitative analysis of a mixture of molecular compounds by two-dimensional gas chromatography, in which a two-dimensional chromatogram (CHR) is generated from a chromatographic signal (SB), and chromatographic peaks are selected at the polygonal aid.

The method comprises the following steps for at least one polygon: adapting a polygon by identifying parts of the chrom.atographic signal contained in the polygon, with the following further steps: (a) determining start times, end times, and maximum chromatographic peaks which are present on the portions. (b) The polygon is set by moving points of intersection between the polygon and the portions as a function of the start times, the end times, and the maximum chromatographic peaks. (c) An amount of at least one molecular compound is determined by calculating the area of the polygon adapted in this way.

WO 1997/01142 A1, which corresponds to U.S. Pat. No. 5,602,755, describes a method for predicting physical, perception, performance, or chemical properties of a complex hydrocarbon mixture, which comprises: (a) Selecting at least one property of the hydrocarbon mixture. (b) Selecting reference samples, wherein the reference samples contain characteristic compound types that are present in the complex hydrocarbon mixture and have the known values of the property or properties selected in step (a). (c) Creating a sample set using the steps: (1) Injecting each reference sample into a gas chromatograph which is connected to a mass spectrometer, thereby causing at least a partial separation of the hydrocarbon mixture into chemical constituents. (2) Introducing the chemical components of each reference sample into the mass spectrometer under dynamic flow conditions. (3) Obtaining a series of time-resolved mass chromatograms for each reference sample. (4) Calibrating the mass chromatograms to correct the retention times. (5) Selecting a series of corrected retention time windows. (6) Selecting a series of molecular and/or fragment ions within each retention time window, wherein the ions are representative of characteristic compounds or molecular classes expected within the retention time window. (7) Recording the total amount of each characteristic compound or compound group selected in step c (6). (8) Creating the data from steps c (6) and c (7) in an X block matrix. (9) Creating the data selected in (a) for reference samples selected in (b) in a Y block matrix. (10) Analyzing the data from steps c (8) and c (9) using multivariate correlation techniques, including partial least squares, main component regression, or ridge regression in order to generate a series of coefficients. (d) Subjecting an unknown hydrocarbon mixture to steps c (I) to c (3) in the same way as the reference sample to generate a series of time-resolved mass chromatograms. (e) Repeating steps c (4) to c (8) for each mass chromatogram of step (d) and (f) Multiplying the matrix from step (e) by the coefficients from step c (10) to generate a predicted value of the property or properties.

In summary, in the prior art according to DE 689 27 133 T2, a (molecular) group formation is proposed, whereas, according to EP 1 953 545 A1, peak groups are determined, wherein, finally, WO 1997/01142 A1 discloses the selecting of molecular and/or fragment ions in order to analyze hydrocarbon mixtures.

### SUMMARY OF THE INVENTION

It is therefore an object of the invention to develop a simplified method for determining a sooting tendency index for a substance mixture, which includes a plurality of individual components, in particular hydrocarbon types and oxygenates, which differ in terms of material and overall volume, wherein the method result is to be suitable for inputting the sooting tendency of substance mixtures to be compared, in the form of a sooting tendency index associated with the substance mixtures, into the standardization.

The starting point of the method according to the invention is the ISO 22854:2016 standard entitled “Liquid petroleum products—Determination of hydrocarbon types and oxygenates in automotive-motor gasoline and in ethanol (E85)—Multidimensional gas chromatography method”.

A reformulyzer analysis according to ISO 222854 exists for most fuels occurring today. There is thus usually no individual component analysis, but rather a reformulyzer analysis according to the ISO 22854 standard. A gas chromatography is carried out according to ISO 22854. The result is then shown as a so-called reformulyzer in a so-called reformulyzer output. In the reformulyzer analysis according to ISO 22854, the individual components are classified into functional supergroups and into reformulyzer groups, wherein the reformulyzer analysis contains the reformulyzer groups i which are distinguished according to the number of carbon atoms. The reformulyzer groups i classified according to the number of carbon atoms are also referred to as reformulyzer subgroups.

Multi-dimensional gas chromatography also provides the percentage volumetric proportion Vᵢ of the respective reformulyzer subgroup i.

To determine the sooting tendency using gas-chromatographic component analysis of hydrocarbon types and oxygenates of a substance mixture, in particular of a fuel (F) or of an additive (B) by means of a reformulyzer analysis, the following steps are provided:
Classifying the hydrocarbon types and oxygenates of the substance mixture determined within the component analysis into functional reformulyzer supergroups,
Classifying the determined hydrocarbon types and oxygenates according to the number of carbon atoms into reformulyzer subgroups (i), which are associated with the functional reformulyzer supergroups,
Determining the percentage volumetric proportion (Vᵢ) of the respective reformulyzer subgroup (i) from the total volume of the substance mixture.

According to the invention, further steps are provided, which
(IV) Assigning a yield sooting index (YSIᵢ) to each reformulyzer subgroup (i) and
(V) Determining a total yield sooting index (YSI_{Total}) according to the equation (1) YSI_{Total}=ΣYSIᵢ·Vᵢ/100, wherein the volumetric proportion (Vᵢ) of each reformulyzer subgroup (i) is multiplied by that of the yield sooting index (YSIᵢ) associated with the reformulyzer subgroup (i), according to which the determined values are summated to the total yield sooting index (YSI_{Total}).

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a general procedure in a schematic overview representation, and

FIGS. 2 and 3 show a reformulyzer analysis carried out according to ISO 222854.

### DETAILED DESCRIPTION

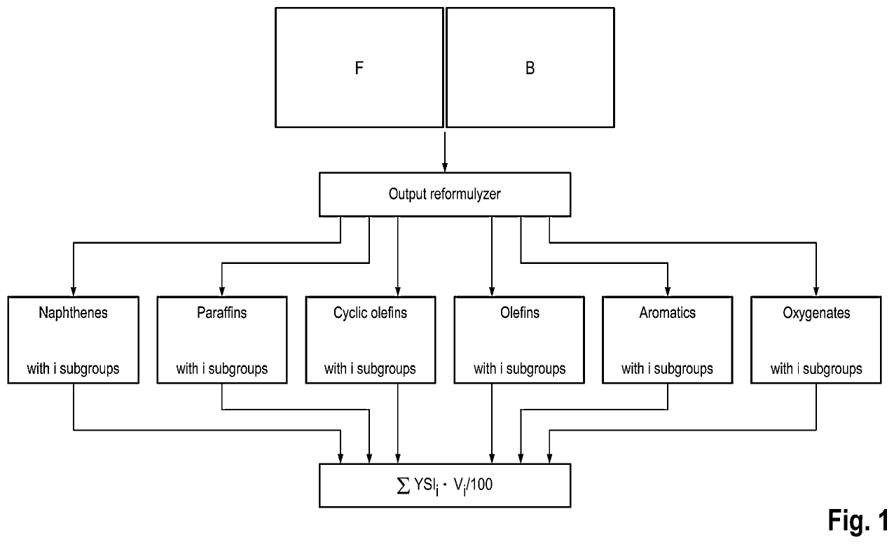

FIG. 1 shows the basic procedure in a schematic overview representation.

By means of the method according to the invention, gasoline engine fuels F or additives B produced on the basis of hydrocarbons, so-called blends, which are mostly added to the gasoline engine fuels F as additives, are examined in terms of their sooting tendency.

Within the method, a reformulyzer analysis carried out according to ISO 222854, as explained and illustrated in FIG. 1 to 3, provides a classification of the individual components into functional reformulyzer supergroups and into reformulyzer subgroups i, wherein, within the reformulyzer analysis according to ISO 222854, the percentage volumetric proportion Vᵢ of the respective reformulyzer subgroup i is determined, and is thus available to further develop the method.

The functional reformulyzer supergroups are naphthenes, paraffins, cyclic olefins, olefins, aromatics, and oxygenates.

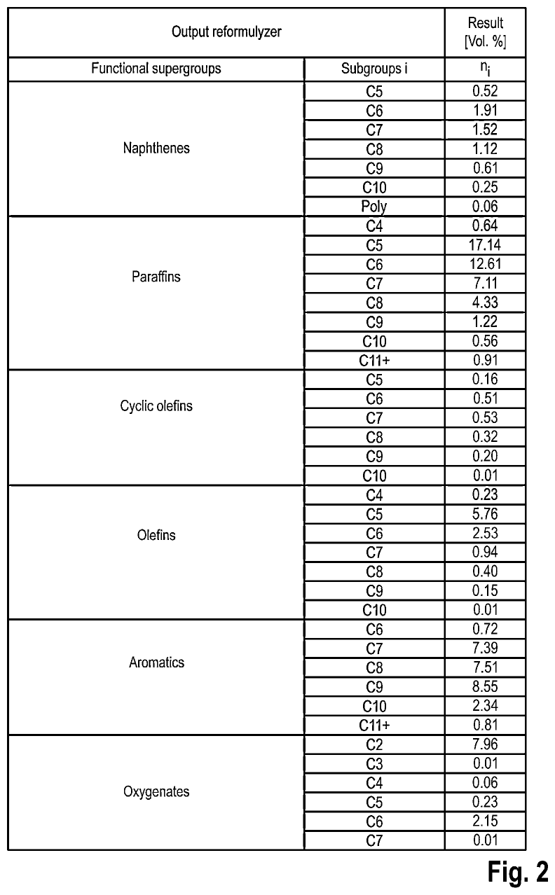

According to FIG. 2, the reformulyzer subgroups i are associated with the respective functional supergroups and sorted according to quantity with the reference sign Vᵢ (V=volumetric proportion in % of the total volume and i=number of carbon atoms) of the carbon atoms.

According to the invention, each of these reformulyzer subgroups i is associated with a yield sooting index which is subsequently referred to as a YSIᵢ and provided with the reference sign YSIᵢ. It is therefore also possible to refer to a YSIᵢ subgroup.

In the reformulyzer analysis according to ISO 222854, only the group classification i and the percentage volumetric proportion Vᵢ are available for the individual components but the yield sooting index of the individual components are not, so that ultimately no value can be formed for a yield sooting index YSIᵢ of a reformulyzer subgroups i either.

The method according to the invention overcomes this disadvantage as follows:
The yield sooting index YSIᵢ of the respective reformulyzer subgroup i is taken from a database available for this purpose, which database the applicant created by evaluating the individual components belonging to the respective reformulyzer subgroups i.
In other words, the database comprises the individual components belonging to a reformulyzer subgroup i with the respective YSIᵢ of the respective individual components, wherein, according to the invention, the determination of a=YSIᵢ for the respective reformulyzer subgroup i is carried out within the database.
In terms of the database, this YSIᵢ associated with the respective reformulyzer subgroup i applies independently of the percentage volumetric proportion Vᵢ of the total volume of the substance mixture investigated, wherein the percentage volumetric proportion Vᵢ is advantageously provided by the reformulyzer analysis according to ISO 222854.
Different method variants are proposed for defining the YSIᵢ to be used. The following method variants for determining the YSIᵢ represent the YSIᵢ of the reformulyzer subgroup i in various ways, wherein the method variants differ in particular with regard to the effort spent in determining the YSIᵢ of the respective reformulyzer subgroup i. All procedures result in success, but differ in terms of the data set that is taken from the database and made available, as is explained below with reference to the description of the method variant.

Principle: According to the invention, a representative yield sooting index is associated with each of these reformulyzer subgroups i.

Depending on the creation of the representative yield sooting index, the invention proposes the following preferred method variants:

Creation of a representative yield sooting index YSIᵢ-M per reformulyzer subgroup i on the basis of the YSIᵢ values of the individual components of the respective reformulyzer subgroup i taken from the database in a first method variant:

From their own DHA analyses with respect to the individual components of the respective reformulyzer subgroups i and to databases that are unknown to the applicant, the applicant detected the YSIᵢ values of the respective individual components of the respective reformulyzer subgroups i.

For example, a reformulyzer subgroup i=C10 in the reformulyzer supergroup of the aromatics contains=35 different individual components. Within the reformulyzer subgroup i=C10, each individual component is associated with a YSIᵢ value.

To simplify the method, an arithmetic mean value is created from the database YSIᵢ values of the individual components of the reformulyzer subgroup i and is associated with the reformulyzer subgroup i as a representative YSIᵢ-M, which is used as a YSIᵢ in the calculation of the total yield sooting index YSI_{Total} explained below.

Creation of a representative yield sooting index YSIᵢ-Mid per reformulyzer subgroup i on the basis of the YSIᵢ values of the individual components of the respective reformulyzer subgroup i taken from the database in a second method variant:

The starting position, with regard to the available YSIᵢ values from their own DHA analyses with respect to the individual components of the respective reformulyzer subgroups i and databases that are unknown to the applicant, corresponds to the first method variant.

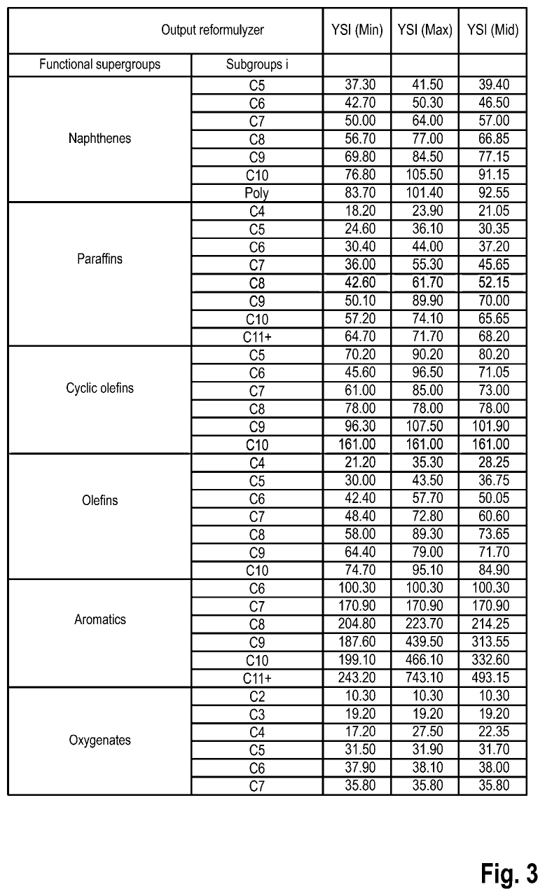

In contrast to the first method variant, it is provided that the representative yield sooting index is an average yield sooting index YSIᵢ-Mid (cf. first of all FIG. 3), which is determined by calculating the arithmetic mean value between a minimum YSIᵢ-Min value and a maximum YSIᵢ-Max value of all individual components of a reformulyzer subgroup i. In the third column of FIG. 3, the associated average yield sooting index mean value YSIᵢ-Mid is recorded, which is used as a YSIᵢ in the calculation of the total yield sooting index YSI_{Total} explained below.

Thus, as is illustrated in FIG. 3, for example, the reformulyzer subgroup i=C10 in the reformulyzer supergroup aromatics can contain=35 different individual components. The YSIᵢ-Min value=199.1 and YSIᵢ-Max value=466.1, whereby in this example, the representative average yield sooting index mean value YSIᵢ-Mid for the reformulyzer subgroup i=C10 results in YSIᵢ-Mid=332.60.

The average yield sooting index YSIᵢ-Mid is associated with the reformulyzer subgroup i as a representative YSIᵢ-Mid, which is used as a YSIᵢ in the calculation of the total yield sooting index YSI_{Total} explained below.

Creation of a representative yield sooting index YSIᵢ-GewM per reformulyzer subgroup i on the basis of the YSIᵢ values of the individual components of the respective reformulyzer subgroup i taken from the database in a third method variant:

The starting position with regard to the available YSIᵢ values from their own DHA analyses with respect to the individual components of the respective reformulyzer subgroups i and to databases that are unknown to the applicant corresponds to the first and second method variants.

According to the invention, in this third method variant, starting from the first method variant of the arithmetic averaging, a weighted arithmetic averaging is provided for the individual reformulyzer subgroups i.

For this purpose, it is/was detected in the database how often a specific individual component occurs in different substance mixtures investigated, in particular fuels F and additives B.

The applicant uses a large database with the yield sooting indices associated with the individual components from at least 60 different conventional fuels F and additives B in the field, i.e., in use, whereby the relevance with respect to the frequency of the respective individual component within a reformulyzer subgroup i can be verified. The respective individual components are evaluated as frequently occurring and thus relevant if they occur in at least half of the fuels under consideration.

As a result, it is advantageously possible to weight the individual components within a reformulyzer subgroup i with respect to their frequency.

There are 35 individual components, for example, in a functional reformulyzer supergroup, for example the aromatics.

For the weighted arithmetic averaging, the frequency values resulting from the database detection are associated with the individual components and taken into consideration in the calculation of the representative weighted arithmetic averaging YSIᵢ-GewM.

In other words, rarely occurring individual components which occur in less than fifty percent of the fuels previously evaluated in the database are thus not weighted during the determination of the weighted arithmetic mean value so that the representative weighted average arithmetic yield sooting index YSIᵢ-GewM differs from the other yield sooting indices, the YSIᵢ-M and YSIᵢ-Mid of the first and second method variants.

The subject matter of this patent application is thus the simplified general method for determining a standardized sooting tendency of a fuel F or an additive B or of a total yield sooting index with the reference sign YSI_{Total} for a fuel F or an additive B and also the manner of determining the representative yield sooting index for the respective reformulyzer subgroups i.

For example, FIG. 2 shows the volumetric proportions Vᵢ of the reformulyzer subgroups i, which are used to calculate the total yield sooting index with the reference sign YSI_{Total} according to Equation 1.

$${YSI}_{Total} = \sum {YSI}_{i} \cdot {V}_{i} / 100 \qquad ( Eq . 1 )$$

YSI_{Total}→Total yield sooting index of the fuel F or of the additive B
YSIᵢ→the YSIᵢ of the respective subgroup i
Vᵢ→the determined volume proportion Vᵢ in percent of the respective subgroup i

Equation 1 provides the general solution to the total yield sooting index YSI_{Total} again.

Depending on the method variant, one of the indices of the YSIᵢ-M or YSIᵢ-Mid or YSIᵢ-GewM explained above is taken into consideration as a YSIᵢ of the respective reformulyzer subgroup i, that is to say, is used in Equation 1.

Various fuels F and/or additives B can thus be compared in a simpler manner than hitherto via the total yield sooting index YSI_{Total}, wherein a higher value of the total YSI characterizes a fuel F or additive B that has a greater tendency to sooting.

The simplification of this procedure includes, in particular, that, a conventional complex DHA evaluation (DHA=Detailed Hydrocarbon Analysis) of all individual components (quantity>350) of the respective substance mixture, in particular of a fuel F and/or of an additive B, no longer has to be determined for a fuel F or an additive B.

In an advantageous manner, this procedure results in a summation over all >350 individual components no longer being necessary; instead only the summation of the subgroups i is necessary taking into account the percentage volumetric proportion Vᵢ of the respective reformulyzer subgroup i.

In addition, by using the reformulyzer or the output of the reformulyzer analysis as a basis for the evaluation, the problem of the many unknowns, as explained above, advantageously no longer occurs.

Finally, a more robust determination is thereby possible, wherein the result can be achieved with significantly less effort, as is clear from the previous description.

In a first example variant, it is provided that functional reformulyzer supergroups are additionally combined. For example, the functional reformulyzer supergroups of the olefins and the cyclic olefins are combined to form a functional reformulyzer supergroup. As a result, fewer data have to be summed in the last step of the method, the summating.

According to a second example variant, reformulyzer subgroups i which are close to one another and the values of which are less than five volume percent apart are combined within a functional reformulyzer supergroup.

For example, within a functional reformulyzer supergroup, the reformulyzer subgroups i, for example C7 and C8, can be combined into a single reformulyzer subgroup. As a result, fewer data have to be summed in the last step of the method, the summating.

According to a third example variant, reformulyzer subgroups i which are close to one another and the values of which are less than five volume percent apart are combined into a plurality of functional reformulyzer supergroups. Thus, reformulyzer subgroups i can be combined into a reformulyzer subgroup i across a plurality of functional reformulyzer supergroups, for example C7 and C8 to C(7+8) of the reformulyzer supergroups. As a result, fewer data have to be summed in the last step of the method, the summating.

In a fourth example variant, it is provided that reformulyzer subgroups i which are close to one another and the values of which are less than five volume percent apart of all functional reformulyzer supergroups are combined. For example, it is possible to combine across all functional reformulyzer supergroups, for example C7 and C8 to C(7+8) of all reformulyzer supergroups, to create a reformulyzer subgroup i. As a result, fewer data have to be summed in the last step of the method, the summating.

The first example variant can advantageously be combined with at least one of the second to fourth examples.

In an advantageous manner, the “combining” according to the explained example variants reduces the effort of calculating the total yield sooting index YSI_{Total} as a function of the selected example variant YSI_{Total} accordingly.

In summary, the advantages of the method are that the costs for determining the total yield sooting index YSI_{Total} are reduced since no individual component analysis has to be carried out and existing standards (ISO 22854) can be used. Since unknowns no longer occur when using the results of the reformulyzer analysis, as an advantage of the method a significantly more robust result is also achieved, which does not fail because of unknown input variables, meaning that the result can always be determined. Moreover, according to the method, a standard determination of total YSI index YSI_{Total} results advantageously in a standard and standardized statement regarding the sooting tendency for each fuel F or additive B. This means that by using the total yield sooting index YSI_{Total}, a comparison of the total yield sooting indices YSI_{Total} of different fuels F and additives B can advantageously be performed and standardized in a standard.

Finally, according to the invention, all fuels in which the sooting tendency has to be predicted can be examined and evaluated, in particular standardized, by means of this method. In an advantageous manner, the method can already be applied during the new development of fuels F or additives B and also in the testing of supply fuels.

In the operation of the vehicles, it is possible according to the invention for the total yield sooting index YSI_{Total} to be transmitted either automatically (for example wirelessly) or manually to a receiving unit, in particular a control device with the receiving unit of a vehicle. If such a value is present in the engine control unit, the yield sooting index YSI_{Total} can be used for more precise determination of the control of the operating parameters of the internal combustion engine and/or of the exhaust system. The yield sooting index YSI_{Total} can also be displayed to the user via the HMI so that the user is provided with standardized information about whether they use a fuel F or an additive B which has a high sooting tendency or a low sooting tendency.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for determining a sooting tendency using gas-chromatographic component analysis of hydrocarbon types and oxygenates of a substance mixture, in particular of a fuel or of an additive, via a reformulyzer analysis, the method comprising:
classifying the hydrocarbon types and oxygenates of the substance mixture determined within the component analysis into functional reformulyzer supergroups;
classifying the determined hydrocarbon types and oxygenates according to the number of carbon atoms into reformulyzer subgroups, which are associated with the functional reformulyzer supergroups;
determining a percentage volumetric proportion of a respective reformulyzer subgroup from a total volume of the substance mixture;
assigning a yield sooting index to each reformulyzer subgroup;
determining a total yield sooting index according to the equation: YSI_{Total}=ΣYSIᵢ·Vᵢ/100,
wherein the volumetric proportion (Vᵢ) of each reformulyzer subgroup (i) is multiplied by that of the yield sooting index (YSIᵢ) associated with the reformulyzer subgroup (i), according to which determined values are summated to the total yield sooting index (YSI_{Total}).

2. The method according to claim 1, wherein that the yield sooting index associated with the respective reformulyzer subgroup (i) (YSIᵢ) is taken from a database, wherein the value of the yield sooting index (YSIᵢ) of the respecting reformulyzer subgroup (i) is provided on the basis of the respective individual components of a reformulyzer subgroup (i) available in the database.

3. The method according to claim 2, wherein the yield sooting index (YSIᵢ) is created from the database YSIᵢ values of the individual components of the reformulyzer subgroup (i) as an arithmetic mean, wherein, in the respective arithmetic mean, either:
values of the yield sooting indices (YSIᵢ) of all individual components of the reformulyzer subgroup (i) flow into a representative arithmetic mean value (YSIᵢ-M); or
a minimum YSIᵢ value (YSIᵢ-Min) and a maximum YSIᵢvalue (YSIᵢ-Max) of the yield sooting indices (YSIᵢ) of all individual components of the reformulyzer subgroup (i) flow into a representative arithmetic mean value (YSIᵢ-Mid); or
values of the yield sooting indices (YSIᵢ) of all individual components of the reformulyzer subgroup (i) flow into a representative arithmetic mean value (YSIᵢ-GewM), which weighs the frequency of occurrence of the individual components within the reformulyzer subgroup (i), wherein only individual components which occur in at least fifty percent of the fuels considered in the database are taken into account during the weighting.

4. The method according to claim 1, wherein, prior to determining the total yield sooting index (YSI_{Total}), at least two or more functional reformulyzer supergroups are combined into a functional reformulyzer supergroup.

5. The method according to claim 1, wherein, prior to determining the total yield sooting index (YSI_{Total}), reformulyzer subgroups (i) that are close to one another within a functional reformulyzer supergroup, the values of which are less than five volume percent apart, are combined.

6. The method according to claim 1, wherein, prior to determining the total yield sooting index (YSI_{Total}) reformulyzer subgroups (i) that are close to one another of a plurality of functional reformulyzer supergroups, the values of which are less than five volume percent apart, are combined.

7. The method according to claim 1, wherein, prior to determining the total yield sooting index (YSI_{Total}), reformulyzer subgroups (i) that are close to one another of all functional reformulyzer supergroups, the values of which are less than five volume percent apart, are combined.

8. The method according to claim 4, wherein, the combining of functional reformulyzer supergroups to form a functional reformulyzer supergroup is combined with combining reformulyzer subgroups (i) that are close to one another, the values of which are less than five volume percent apart.

9. The method according to claim 1, wherein, the yield sooting index (YSI_{Total}) is transmitted either automatically or manually to a receiving unit of a vehicle.

10. The method according to claim 9, wherein, the yield sooting index (YSI_{Total}) is used as an additional input variable for more precise determination of the control of the operating parameters of an internal combustion engine and/or an exhaust system of the vehicle.

11. The method according to claim 1, wherein, the total yield sooting index (YSI_{Total}) is used for standardization with regard to the sooting tendency of a plurality of, at least two, different substance mixtures investigated within the method, in particular fuels or additives.
